(19) United States
(12) Patent Application Publication
(10) Pub. No.: US 2020/0224279 A1
(43) Pub. Date: Jul. 16, 2020

(54) CO-ASSAYS TO FUNCTIONAL CANCER BIOMARKER ASSAYS

(71) Applicants: Massachusetts Institute of Technology, Cambridge, MA (US); Dana-Farber Cancer Institute, Inc., Boston, MA (US)

(72) Inventors: David Weinstock, Jamaica Plain, MA (US); Keith Ligon, Brookline, MA (US); Scott R. Manalis, Portland, OR (US); Mark Murakami, Cambridge, MA (US); Mark M. Stevens, Cambridge, MA (US)

(73) Assignees: Massachusetts Institute of Technology, Cambridge, MA (US); Dana-Farber Cancer Institute, Inc., Boston, MA (US)

(21) Appl. No.: 16/739,820

(22) Filed: Jan. 10, 2020

Related U.S. Application Data
(60) Provisional application No. 62/790,746, filed on Jan. 10, 2019.

(51) Int. Cl. C12Q 1/6886; G16H 50/30; G16B 20/20

(52) U.S. Cl. CPC C12Q 1/6886; C12Q 2600/156; G16B 20/20; G16H 50/30

(57) ABSTRACT

The invention provides methods for evaluating disease, such as cancer, by way of performing multiple assays involving single-cell analysis on live cells isolated from a sample of a patient. The data obtained from the multiple assays is analyzed and linked to thereby provide a characterization of any given cell having undergone analysis, which, in turn, allows for evaluation of the sample either known to be, or suspected of being, cancerous. A report may be generated based on the data analysis, wherein the report provides information related to the cancer evaluation, including, but not limited to, whether the sample tested positive for cancer, a determination of a stage or progression of cancer, and a customized treatment plan tailored to an individual patient's cancer diagnosis.

### RELATED APPLICATION

This application claims priority under 35 U.S.C. § 119(e) to U.S. Provisional Application Ser. No. 62/790,746, filed Jan. 10, 2019, the entire contents of which are incorporated by reference herein.

### TECHNICAL FIELD

The disclosure relates to methods for evaluating disease.

### BACKGROUND

Cancer is a global health issue that causes millions of deaths worldwide every year. Standard treatments typically are based on the evaluation of cell lines, animal models, and human subjects. Still, individual patient response to a drug or therapy are often variable and unpredictable even for cancers of identical tissue origin and common histology. Consequently, while current treatments benefit some patients, other patients may receive little to no benefit and may further suffer from adverse reactions. Accordingly, while there are many different cancer treatments available, there is limited ability to effectively predict how an individual patient will respond to a particular treatment, which may lead to extended periods of time in which a patient endures a treatment that simply isn't working as intended.

### SUMMARY

Methods of the invention include multiple co-assays that are performed on individual living cells obtained from a patient. The co-assays preferably include both functional and genomic tests and are useful for evaluating therapeutic choice and potential patient response in any disease, but have particular application in cancer. Methods of the disclosure measure a functional property of a live cell, while leaving the cell available for other assays, which may be further functional assays or may include genomic assays such as genome sequencing. The functional properties may include growth, stagnation, or atrophy of living cells indicative of disease state or drug response. After measuring growth or atrophy of living cells, genomic data can be obtained from that same living cell, giving clinicians precise methodology for personalized medicine. In preferred embodiments, a tissue or fluid sample is obtained from a patient and a cell from the sample is loaded into a functional measurement instrument. The instrument is operated to measure a property such as mass or mass change of the living cell, and that living cell is provided to another instrument such as a next-generation sequencing instrument to sequence genomic material or a set of cancer-associated markers. A positive mass accumulation rate is indicative of a malignant, transformed cell and genomic features reported by the NGS instrument may be correlated to disease status. Instruments for use in the invention provide rapid, high-throughput measurements. Moreover, since functional properties and genomic features are taken from the same cell, methods of the invention provide the ability to correlate genetic markers of cancer to disease status and/or drug response with great sensitivity and specificity. Thus, methods and instruments of the invention provide personalized diagnostic and prognostic information.

According to the invention, diseases, such as cancer, are analyzed using multiple assays involving single-cell analysis on live cells isolated from a patient sample. The data obtained from the multiple assays are analyzed and linked to provide a characterization of any given cell having undergone analysis which, in turn, allows for evaluation of the sample to identify diseases cells and their associated genetic markers.

A report may be generated based on the data analysis to provide diagnostic information relative to stage or progression and therapeutic choice, resulting in a customized treatment plan tailored to an individual patient's cancer diagnosis.

Methods of the present invention involve performing multiple assays involving single-cell analysis on live cells isolated from a patient. In a preferred embodiment, methods of the invention are used to diagnose cancer by the presence of mass accumulation and/or genomic or epigenomic markers indicative of cancer. Preferred methods include performing an initial assay on live cells to obtain a functional biomarker measurement of one or more live cells, such as single-cell biophysical properties, including, but not limited to, mass, growth rate, and mass accumulation of an individual living cell. The initial assay may generally be performed with any functional biomarker measurement instrument, such as, for example, an instrument comprising a suspended microchannel resonator (SMR) or serial SMR (sSMR). The SMR may be used to precisely measure biophysical properties, such as mass and mass changes, of a single cell flowing therethrough. Mass change may be expressed as mass accumulation rate (MAR). When used with cancer cells, those changes provide a functional, universal biomarker by which medical professionals (e.g., oncologists) may monitor the progression of a cancer and determine how cancer cells respond to therapies.

The SMR provides a sensitive “scale” that measures small changes in mass of a single cell. When cancer cells respond to cancer drugs, mass change indicative of cell death begins very quickly. The SMR can detect the small changes in mass that indicate that cells are dying. The speed and sensitivity allow the SMR to detect a cancer cell's response to a cancer drug while the cell is still living. Upon flowing the live cells through the SMR, a functional biomarker, such as mass or MAR, in the one or more live cells is obtained. The MAR measurements also characterize heterogeneity in cell growth across cancer cell lines. Individual live cells are able to pass through the SMR, wherein each cell is weighed multiple times over a defined interval. The SMR includes multiple sensors that are fluidically connected (e.g., in series) and separated by delay channels. Such a design enables a stream of cells to flow through the SMR such that different sensors can concurrently weigh flowing cells in the stream, revealing single-cell MARs. The SMR is configured to provide real-time, high-throughput monitoring of mass change for the cells flowing therethrough. Therefore, the biophysical properties, including mass and/or mass changes (e.g., MAR), of a single cell can be measured.

Upon passing through the functional biomarker measurement instrument, the single cells remain viable and can be isolated downstream from the instrument where the cells may undergo subsequent use, such as testing in traditional assays.

Methods further include performing one or more additional assays on the live cells, either concurrently with the initial assay or later, to obtain further data. The one or more additional assays may include, for example, genome sequencing. In order to perform sequencing, methods of the disclosure further include extracting nucleic acid from the one or more live cells having undergone the first analysis for downstream genomic sequencing assay. Isolation, extraction or derivation of genomic nucleic acids may be performed by methods known in the art. For example, isolating nucleic acid from a live single cell generally includes treating a cell in such a manner that genomic nucleic acids present in the cell are extracted and made available for analysis, such as lysing the cells to isolate genomic nucleic acid. Sequencing the one or more live cells may be by any method known in the art and sequencing produces a plurality of sequence reads. The sequence reads can be analyzed to detect and describe variations, including, but not limited to, structural abnormalities, copy number variants, microdeletions, or duplications.

Results of the multiple assays, specifically the data obtained from the first assay (i.e., single-cell functional biomarker measurements, such as mass accumulation rate) and data obtained from the one or more additional assays (e.g., single-cell genetic data), are then analyzed to thereby provide a characterization of any given cell, in turn allowing for detailed evaluation of the patient sample. In particular, data analysis may include linking the biophysical measurements with sequence read data of the same cell, which may allow for characterization of an underlying transcriptional program associated with cellular mass and growth rate variability in a range of normal and dysfunctional biological contexts. In turn, a report may be generated based on the data analysis, wherein the report provides information related to the cancer evaluation, including, but not limited to, whether the sample tested positive for cancer, a determination of a stage or progression of cancer, and a customized treatment plan tailored to an individual patient's cancer diagnosis. As such, the methods of the present invention improve outcomes of cancer treatment, avoid any unnecessary cancer treatment, and reduce overall healthcare costs.

Accordingly, methods of the present invention allow for multiple assays to be performed on a sample of live cells, thereby providing real-time morphological and phenotypic insight of such cells. In particular, the initial assay is performed on an instrument including a suspended microchannel resonator (SMR), which has an exquisitely sensitive scale that can measure small changes in mass of a single cell. When cancer cells respond to cancer drugs, such cells start their process of dying by changing mass within hours. The speed and sensitivity of the SMR enable the SMR to detect a cancer cell's response (i.e., changes in mass) to therapies while the cell is still living, wherein such responses are not discernable via genomic measurements and can only be obtained on live cells. Such responses provide a functional, universal biomarker by which medical professionals may monitor the progression of a cancer and determine how cancer cells respond to therapies. In particular, the biophysical properties (i.e., mass, change in mass, and MAR) offer unique insights into a wide range of biological phenomena of a live cancer cell, including, but not limited to, basic patterns of single-cell mass and growth regulation, biophysical changes associated with immune cell activation, and cancer cell heterogeneity in the presence or absence of a drug therapy.

Furthermore, single cells remain viable after SMR measurement. Those same cells may be analyzed post-SMR to produce data that are complementary to the SMR measurement. As such, data from the additional assays are processed with SMR data to provide a more comprehensive diagnostic and prognostic analysis. The accumulated data are presented as a report that may provide an initial diagnosis and/or information on staging, classification, or recurrence. Clinicians may use the report to create an individualized treatment plan. As such, the methods of the present invention can improve outcomes of cancer.

Aspects of the invention are accomplished by obtaining live cells isolated from a sample of patient, such as a tumor or a bodily fluid, either known to be or suspected as being cancerous, and performing single-cell analysis on the live cells. The live cells may include at least one of a cancer cell and a cancer-related immune cell, such as a lymphocyte for example. The live cells undergo a first assay to obtain a functional property of the live cells, specifically a functional biomarker measurement. In particular, the first assay involves loading individual live cells into a functional biomarker measurement instrument, such as, for example, a suspended microchannel resonator (SMR) measurement instrument and flowing the live cells through the SMR. The SMR may be used to precisely measure biophysical properties, such as mass and mass changes, of a single cell flowing therethrough. The mass change may be mass accumulation rate (MAR). The live cells remaining in a living state upon passing through the SMR instrument, such that they are accessible for one or more additional live cell assays downstream from the first assay. The method further includes performing at least a second assay on the live cells to obtain additional data. The second assay is performed on the live cells having undergone the first assay, which allows for data obtained from the first and second assays to be linked at a single-cell level, as opposed to a population level. The method further includes analyzing data from the second assay and the measured cancer biomarker from the first assay to determine a stage or progression of cancer.

In some embodiments, the second assay is selected from the group consisting of genome sequencing, single cell transcriptomics, single cell proteomics, and single cell metabolomics. As such, performing the second assay may include sequencing nucleic acid from the one or more live cells having undergone the first assay to produce sequence data and the analyzing step may include analyzing the sequence data. In turn, one or more polymorphisms in the sequence data may be detected. Additionally, or alternatively, the analyzing step may include mapping unique sequence reads to a reference to determine sub-chromosomal copy number variation or aneuploidy. Additionally, or alternatively, the analyzing step may include determining expression levels in the one or more live cells. In some embodiments, the method further comprises providing a report that describes one or more genetic sequence alterations and the measured cancer biomarker in the live cells from the patient.

In some embodiments, the analyzing step may further include determining tumor mutational burden (TMB). The TMB may be is determined by mapping sequence reads to a reference genome, identifying differences between the reads and the reference, and adding the identified difference to a mutation count. As previously noted, in some embodiments, the functional cancer biomarker measured in the first assay may include mass and/or mass change, wherein the functional cancer biomarker may be measured after administration of a checkpoint inhibitor. Yet still, in some embodiments, the functional cancer biomarker may include a mass or change in mass of a live cancer-related immune cell isolated from the same sample as the live cancer cell, such that the method may further include correlating the cancer-related immune cell biophysical data with the TMB data of the live cancer cell to generate composite biomarker indicating a stage or progression of the cancer.

In some embodiments, the analyzing step includes analyzing sequence data from a plurality of different cells from a sample from the patient, assigning the cells to clonal groups based on the sequence data, and measuring the functional cancer biomarker for cells from specific clonal groups. In some embodiments, the functional cancer biomarker measured in the first assay may include a mass accumulation rate. As such, in some embodiments, the method further includes identifying mutations exclusively present in clonal groups with the highest mass accumulation rate(s) as putative driver mutations. In some embodiments, the method further includes identifying mutations whose presence does not correlate with mass accumulation rate as passenger mutations.

### BRIEF DESCRIPTION OF THE DRAWINGS

FIG. 1 diagrams a method for disease evaluation.

FIG. 2 shows an instrument for measuring a functional property of a living cell.

FIG. 3 shows a suspended microchannel resonator (SMR) device.

FIG. 4 shows a serial suspended microchannel resonator (sSMR) array.

FIG. 5 diagrams an SMR detection system of the disclosure.

FIG. 6 diagrams a sequencing workflow consistent with the present disclosure.

FIG. 7 shows a report as may be provided.

FIG. 8 is a block diagram of a system consistent with the present disclosure.

### DETAILED DESCRIPTION

The invention provides methods for evaluating disease, such as cancer, by way of performing multiple assays involving single-cell analysis on live cells isolated from a sample of a patient, wherein the sample is either known to have, or suspecting of having, cancer cells or cancer-related cells (e.g., immune cells). The data obtained from the multiple assays is analyzed and linked to thereby provide a characterization of any given cell having undergone analysis, which, in turn, allows for evaluation of the sample either known to be, or suspected of being, cancerous. A report may be generated based on the data analysis, wherein the report provides information related to the cancer evaluation, including, but not limited to, whether the sample tested positive for cancer, a determination of a stage or progression of cancer, and a customized treatment plan tailored to an individual patient's cancer diagnosis.

The methods of the present invention allow for multiple assays to be performed on a sample of live cells, thereby providing real-time morphological and phenotypic insight of such cells. In particular, the initial assay is performed on an instrument including a suspended microchannel resonator (SMR), which has an exquisitely sensitive scale that can measure small changes in mass of a single cell. When cancer cells respond to cancer drugs, such cells start their process of dying by changing mass within hours. The speed and sensitivity of the SMR enable the SMR to detect a cancer cell's response (i.e., changes in mass) to therapies while the cell is still living, wherein such responses are not discernable via genomic measurements and can only be obtained on live cells. The cancer cell's responses provide a functional, universal biomarker by which medical professionals may monitor the progression of a cancer and determine how cancer cells respond to therapies. In particular, the biophysical properties (i.e., mass, change in mass, and MAR) offer unique insights into a wide range of biological phenomena of a live cancer cell, including, but not limited to, basic patterns of single-cell mass and growth regulation, biophysical changes associated with immune cell activation, and cancer cell heterogeneity in the presence or absence of a drug therapy.

The single cells remain viable upon passing through the SMR instrument and can further be isolated downstream from the instrument where the cells may undergo subsequent assays to obtain additional measurements of the one or more live cells, such as genetic data. As such, data from the additional assays can be analyzed with data from the initial assay, to thereby provide a detailed characterization of any given cell, in turn allowing for a more comprehensive cancer evaluation of the patient sample. A report may be generated based on the data analysis, wherein the report provides information related to the cancer evaluation, including, but not limited to, whether the sample tested positive for cancer, a determination of a stage or progression of cancer, and a customized treatment plan tailored to an individual patient's cancer diagnosis. As such, the methods of the present invention can improve outcomes of cancer treatment, avoid any unnecessary cancer treatment, and reduce overall healthcare costs.

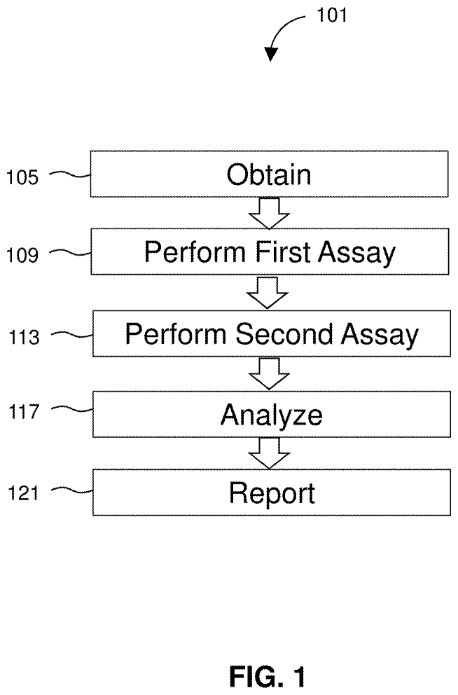

FIG. 1 diagrams a method 101 for evaluating a disease. The method 101 includes obtaining 105 one or more live cells isolated from a sample of patient. The method 101 further includes performing 109 a first assay on the one or more live cells. The first assay includes measuring a functional cancer biomarker in the one or more live cells. For example, in one embodiment, the functional cancer biomarker includes single-cell biophysical properties, including, but not limited to, mass, growth rate, and mass accumulation of an individual living cell. In some embodiments, as will be described in greater detail herein, the first assay may generally be performed with any functional biomarker measurement instrument, such as, for example, an instrument comprising a suspended microchannel resonator (SMR) or serial SMR (sSMR). The SMR may be used to precisely measure biophysical properties, such as mass and mass changes, of a single cell flowing therethrough. The mass change may be mass accumulation rate (MAR). When used with cancer cells, those changes provide a functional, universal biomarker by which medical professionals (e.g., oncologists) may monitor the progression of a cancer and determine how cancer cells respond to therapies.

Upon passing through the functional biomarker measurement instrument, the single cells remain viable and can be isolated downstream from the instrument where the cells may undergo subsequent use, such as testing in traditional assays.

The method 101 further includes performing 113 at least a second assay on the live cells, either concurrently with the initial assay, or downstream from the first assay, to obtain further data associated with the live cells, such as additional functional data and/or genomic data. As will be described in greater detail herein, the second assay may include genome sequencing, single cell transcriptomics, single cell proteomics, and single cell metabolomics. Yet still, in other embodiments, the second assay, or an additional assay, may include flow cytometry to analyze physical and/or chemical characteristics of the one or more cells, including the detection of biomarkers.

The method 101 further includes analyzing 117 data from the second assay and the measured cancer biomarker from the first assay to determine at least a stage or progression of cancer. For example, in one embodiment, the second assay includes genome sequencing, wherein sequencing produces sequence reads, which may be analyzed in conjunction with the biophysical measurements (i.e., mass, growth rate, mass accumulation, changes in mass, etc.) to identify clinically-significant information, including a characterization of any given cell, such as characterization of an underlying transcriptional program associated with cellular mass and growth rate variability in a range of normal and dysfunctional biological contexts. Accordingly, results of the multiple assays, specifically the data obtained from the first assay (i.e., single-cell functional cancer biomarker measurements) and data obtained from the one or more additional assays (e.g., single-cell genetic data), are analyzed to thereby provide a characterization of any given cell, in turn allowing for detailed evaluation of the patient sample.

The method 101 further includes providing 121 a report comprising information related to the cancer evaluation, including, but not limited to, specific data associated with the first and second assays, whether the sample tested positive for cancer, a determination of a stage or progression of cancer, and a customized treatment plan tailored to an individual patient's cancer diagnosis.

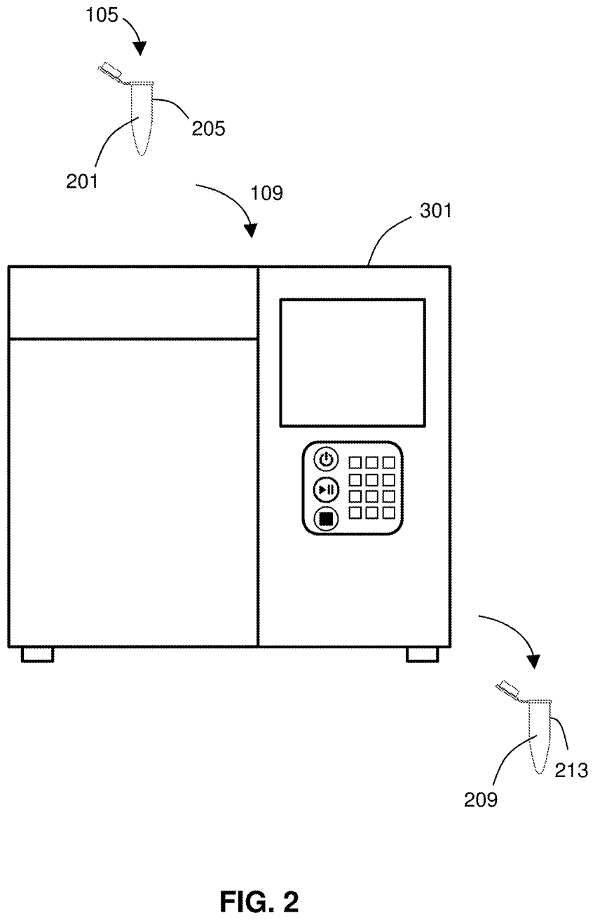

FIG. 2 shows a sample 201 provided within a suitable container 205, wherein the sample 201 includes one or more live cells including at least one of a cancer cell and a cancer-related immune cell obtained 105 from a patient known to have, or suspected of having, cancer. For example, in some embodiments, samples may be collected and stored in their own container, such as a centrifuge tube such as the 1.5 mL micro-centrifuge tube sold under the trademark EPPENDORF FLEX-TUBES by Eppendorf, Inc. (Enfield, Conn.).

The one or more live cells are isolated from a biological sample of a patient known to have, or suspected of having, cancer. A biological sample may include a human tissue or bodily fluid and may be collected in any clinically acceptable manner. For example, the sample may include a fine needle aspirate or a biopsy from a tissue known to be, or suspected of being, cancerous. The sample may include a bodily fluid from a patient either known to include, or suspected of including, cancer cells or cancer-related cells (i.e., immune cells).

A tissue may include a mass of connected cells and/or extracellular matrix material, e.g. skin tissue, hair, nails, nasal passage tissue, CNS tissue, neural tissue, eye tissue, liver tissue, kidney tissue, placental tissue, mammary gland tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues.

A body fluid may be a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CS. A sample also may be media containing cells or biological material. A sample may also be a blood clot, for example, a blood clot that has been obtained from whole blood after the serum has been removed. In certain embodiments, the sample is blood, saliva, or semen collected from the subject.

The isolation of the one or more live cells from the biological sample may be performed via any known isolation techniques and methods for maintaining a viable collection of cells, which may include one or cancer and/or cancer-related immune cells (e.g., lymphocytes includes T-cells and/or B-cells). For example, if the sample is a tissue sample from a tumor or growth suspected of being cancerous, the tissue sample may undergo any known cell isolation, separation, or dissociation techniques which may involve physical methods (i.e., use of mechanical force to break apart cellular adhesions) and/or reagent-based methods (i.e., use of fluid mediums to break apart cellular adhesions). For example, in one embodiment, a tissue sample (i.e., a fine needle aspirate from a tumor) may be disaggregated to produce a suspension of individual live cells to allow for analysis of cells independently. The tissue sample may undergo initial disaggregation by way of application of a physical force alone to break the tissue sample into smaller pieces, at which point the sample may be exposed to proteolytic enzymes that digest cellular adhesion molecules and/or the underlying extracellular matrix to thereby provide single cells within a suspension.

In a preferred embodiment, the obtaining step 105 includes drawing the sample from a solid tumor by fine needles aspiration. A solid tumor may be interrogated via fine needle aspiration to retrieve a cell mass, or tissue sample, that includes cancer cells. Methods may include using a needle, such as a fine-needle aspiration biopsy using a sharp 25-gauge, 1-inch long needle. A suitable needle is the sharp 25-gauge, 1-inch long needle sold under the trademark PRECISION GLUIDE by BD (Franklin Lakes, N.J.). The needle may be attached to a 10 ml aspirating syringe.

The biopsy needle may be passed into a lesion or tumor. Once the tip of the needle is advanced into the lesion, the tumor cells are aspirated. The plunger of the syringe may be pulled and released a few times, allowing the suction force to equilibrate. The needle is withdrawn and a tissue sample or clump of cells is deposited in or on a substrate such as a slide, culture dish, membrane, or other material. In some embodiments, the clump of cells is deposited on a surface within a collection tube or flask, such as a 1.5 mL microcentrifuge tube sold under the trademark EPPENDORF. Each aspirate may be flushed into the flask using culture media, saline, or a maintenance/nutrient media. The aspiration material may be filtered to deposit clumps or samples of tissue on the surface of a filter membrane. The cell mass may be deposited, e.g., on a nitrocellulose membrane and disaggregated using, e.g., proteases such as collagenase and/or displace. Live cells may be washed into a fluidic tube or system with and supported by a suitable media such as a Ham's nutrient mixture. For information, see Rajer, 2005, Quantitative analysis of fine needle aspiration biopsy samples, Radiol Oncol 39(4):269-72, incorporated by reference.

The tissue sample or clump of cells is disaggregated. Any suitable technique may be used to disaggregate the tissue sample/clump of cells. For example, disaggregation may include physical or mechanical disaggregation, chemical disaggregation, proteolytic disaggregation, or any combination thereof. In some embodiments, proteolytic disaggregation is performed using one or more enzymes. Any suitable enzymes may be used. In some embodiments, the tissue sample/clump of cells is washed with and digested by collagenase I and dispase II. The resultant free cells may be held in a suitable nutrient media such as, for example, Ham's F12 Kaighn's Modification medium in presence of 1 mU/mL bovine thyrotropin (TSH), 10 μg/mL human insulin, 6 μg/mL transferrin, and 10-8 M hydrocortisone.

Thus the method 101 may include obtaining 105 a fine needle aspirate tissue sample, from a solid tumor, that includes live cancer cells that are disaggregated (preferably by proteolytic techniques) from any tissue or clump so that individual live cells may be separately addressed, e.g., subjected to a measurement of some functional property of those cells. It should be noted that the reagents selected for assisting in the disaggregating step should keep the cells intact and not kill the cells.

Other methods currently used for single cell isolation include, but are not limited to, serial dilution, micromanipulation, laser capture microdissection, FACS, microfluidics, Dielectrophoretic digital sorting, manual picking, and Raman tweezers. Manual single cell picking is a method is where cells in a suspension are viewed under a microscope, and individually picked using a micropipette, while Raman tweezers is a technique where Raman spectroscopy is combined with optical tweezers, which uses a laser beam to trap, and manipulate cells. Dielectrophoretic (DEP) digital sorting method utilizes a semiconductor controlled array of electrodes in a microfluidic chip to trap single cells in DEP cages, where cell identification is ensured by the combination of fluorescent markers with image observation and delivery is ensured by the semiconductor controlled motion of DEP cages in the flow cell.

Live cells are loaded onto an instrument 301 capable of performing 109 the first assay on the live cells. The instrument 301 measures a functional cancer biomarker in the one or more live cells, such as single-cell biophysical properties, including, but not limited to, mass, growth rate, and mass accumulation of an individual living cell. The initial assay may generally be performed with an instrument 301 comprising a suspended microchannel resonator (SMR). The SMR may be used to precisely measure biophysical properties, such as mass and mass changes, of a single cell flowing therethrough. The mass change may be mass accumulation rate (MAR). When used with cancer cells, those changes provide a functional, universal biomarker by which medical professionals (e.g., oncologists) may monitor the progression of a cancer and determine how cancer cells respond to therapies.

The SMR may comprise an exquisitely sensitive scale that measures small changes in mass of a single cell. When cancer cells respond to cancer drugs, the cells begin the process of dying by changing mass within hours. The SMR can detect this minor weight change. That speed and sensitivity allow the SMR to detect a cancer cell's response to a cancer drug while the cell is still living. Upon flowing the live cells through the SMR, a functional biomarker, such as mass or MAR, in the one or more live cells is obtained. MAR measurements characterize heterogeneity in cell growth across cancer cell lines. Individual live cells are able to pass through the SMR, wherein each cell is weighed multiple times over a defined interval. The SMR includes multiple sensors that are fluidically connected, such as in series, and separated by delay channels. Such a design enables a stream of cells to flow through the SMR such that different sensors can concurrently weigh flowing cells in the stream, revealing single-cell MARs. The SMR is configured to provide real-time, high-throughput monitoring of mass change for the cells flowing therethrough. Therefore, the biophysical properties, including mass and/or mass changes (e.g., MAR), of a single cell can be measured. Such data can be stored and used in subsequent analysis steps, as will be described in greater detail herein.

Upon passing through the instrument 301, single cells remain viable and can be isolated downstream from the instrument 301 and are available to undergo the subsequent assays. As shown, a sample 209 of the one or more live cells having undergone the first assay (i.e., passing through the instrument 301) are collected in a suitable container 213 and are then available to undergo a second assay.

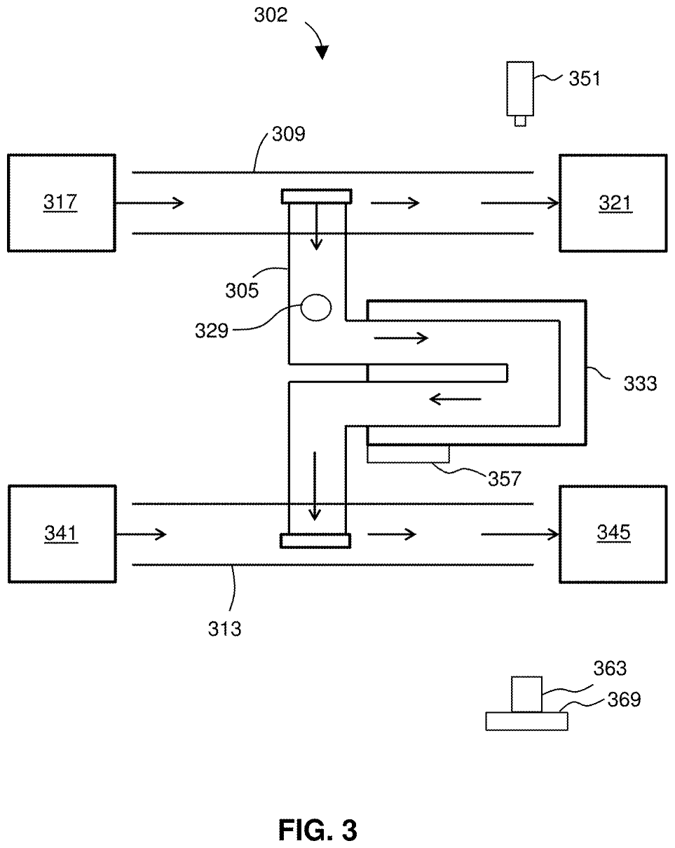

FIG. 3 shows a suspended microchannel resonator (SMR) device 302 of the disclosure. The SMR device 302 includes a microchannel 305 that runs through a cantilever 333, which is suspended between an upper bypass channel 309 and a lower bypass channel 313. Having the two bypass channels allows for decreased flow resistance and accommodates the flow rate through the microchannel 305. Sample eluate 317 flows through the upper bypass channel 309, wherein a portion of the eluate 317 collects in the upper bypass channel collection reservoir 321. A portion of the eluate 317 including at least one live cell 329 flows through the suspended microchannel 305. The flow rate through the suspended microchannel 305 is determined by the pressure difference between its inlet and outlet. Since the flow cross section of the suspended microchannel is about 70 times smaller than that of the bypass channels, the linear flow rate can be much faster in the suspended microchannel than in the bypass channel, even though the pressure difference across the suspended microchannel is small. Therefore, at any given time, it is assumed that the SMR is measuring the eluate that is present at the inlet of the suspended microchannel. The sample includes a live cell or material with cell-like properties.

The cell 329 flows through the suspended microchannel 305. The suspended microchannel 305 extends through a cantilever 333 which sits between a light source 351 and a photodetector 363 connected to a chip 369 such as a field programmable gate array (FPGA). The cantilever is operated on by an actuator, or resonator 357. The resonator 357 may be a piezo-ceramic actuator seated underneath the cantilever 333 for actuation. The cell 329 flows from the upper bypass channel 309 to the inlet of the suspended microchannel 305, through the suspended microchannel 305, and to the outlet of the suspended microchannel 305 toward the lower bypass channel 313. A buffer 341 flows through the lower bypass channel towards a lower bypass channel collection reservoir 345. After the cell 329 is introduced to the lower bypass channel 313, the cell 329 is collected in the lower bypass collection reservoir 345.

In some embodiments, the instrument 301 comprises an array of SMRs with a fluidic channel passing therethrough.

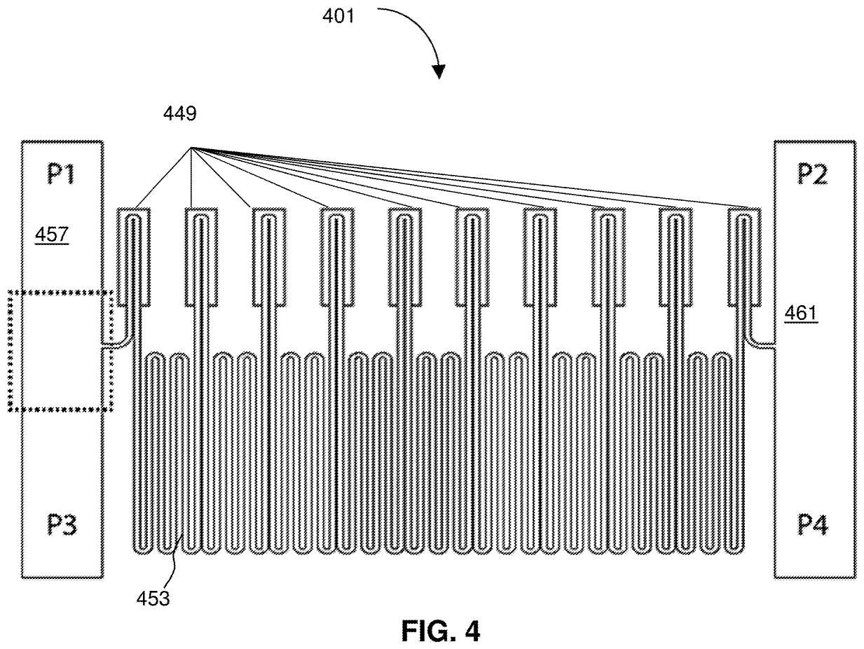

FIG. 4 shows a serial suspended microchannel resonator (sSMR) array 401, made up of an array of SMRs. An instrument that includes an sSMR array is useful for direct measurement of biophysical properties of single cells flowing therethrough. The sSMR includes a plurality of cantilevers 449 and a plurality of delay channels 453. Cells from the first bypass channel 457 through the cantilevers 449 and delay channels 453 to the second bypass channel 461. Pressure differences in the first bypass channel 457 are indicated by P1 and P2, and pressure differences in the second bypass channel 461 are indicated by P3 and P4.

Instruments 301 of the disclosure can make sensitive and precise measurements of mass or change in mass through the use of an sSMR array 401. The instruments use a structure such as a cantilever that contains a fluidic microchannel. Living cells are flowed through the structure, which is resonated and its frequency of resonation is measured. The frequency at which a structure resonates is dependent on its mass and by measuring the frequency of at which the cantilever resonates, the instrument can compute a mass, or change in mass, of a living cell in the fluidic microchannel. By flowing the isolated living cells from the tissue sample through such devices, one may observe the functions of those cells, such as whether they are growing and accumulating mass or not. The mass accumulation or rate of mass accumulation can be related to clinically important property such as the presence of cancer cells or the efficacy of a therapeutic on cancer cells.

Methods for measuring single-cell growth are based on resonating micromechanical structures. The methods exploit the fact that a micromechanical resonator's natural frequency depends on its mass. Adding cells to a resonator alters the resonator's mass and causes a measurable change in resonant frequency. Suspended microchannel resonators (SMRs) include a sealed microfluidic channel that runs through the interior of a cantilever resonator. The cantilever itself may be housed in an on-chip vacuum cavity, reducing damping and improving frequency (and thus mass) resolution. As a cell in suspension flows through the interior of the cantilever, it transiently changes the cantilever's resonant frequency in proportion to the cell's buoyant mass (the cell's mass minus the fluid mass it displaces). SMRs weigh single mammalian cells with a resolution of 0.05 pg (0.1% of a cell's buoyant mass) or better. The sSMR array 401 includes an array of SMRs fluidically connected in series and separated by “delay” channels between each cantilever 349. The delay channels give the cell time to grow as it flows between cantilevers.

Devices may be fabricated as described in Lee, 2011, Suspended microchannel resonators, Lab Chip 11:645 and/or Burg, 2007, Weighing of biomolecules, Nature 446:1066-1069, both incorporated by reference. Large-channel devices (e.g., useful for PBMC measurements) may have cantilever interior channels of 15 by 20 μm in cross-section, and delay channels 20 by 30 μm in cross-section. Small-channel devices (useful for a wide variety of cell types) may have cantilever channels 3 by 5 μm in cross-section, and delay channels 4 by 15 μm in cross-section. The tips of the cantilevers in the array may be aligned so that a single line-shaped laser beam can be used for optical-lever readout. The cantilevers may be arrayed such that the shortest (and therefore most sensitive) cantilevers are at the ends of the array. Before use, the device may be cleaned with piranha (3:1 sulfuric acid to 50% hydrogen peroxide) and the channel walls may be passivated with polyethylene glycol (PEG) grafted onto poly-L-lysine. In some embodiments, a piezo-ceramic actuator seated underneath the device is used for actuation. The instrument 301 may include low-noise photodetector, Wheatstone bridge-based amplifier (for piezo-resistor readout), and high-current piezo-ceramic driver. To avoid the effects of optical interference between signals from different cantilevers (producing harmonics at the difference frequency), the instrument may include a low-coherence-length light source (675 nm super-luminescent diode, 7 nm full-width half maximum spectral width) as an optical lever. After the custom photodetector converts the optical signal to a voltage signal, that signal is fed into an FPGA board, in which an FPGA implements twelve parallel second-order phase-locked loops which each both demodulate and drive a single cantilever. The FPGA may be on a DE2-115 development board operating on a 100 MHz clock with I/O provided via a high-speed AD/DA card operating 14-bit analog-to-digital and digital-to-analog converters at 100 MHz.

To operate all cantilevers in the array, the resonator array transfer function is first measured by sweeping the driving frequency and recording the amplitude and phase of the array response. Parameters for each phase-locked loop (PLL) are calculated such that each cantilever-PLL feedback loop has a 50 or 100 Hz FM-signal bandwidth. The phase-delay for each PLL may be adjusted to maximize the cantilever vibration amplitude. The FM-signal transfer function may be measured for each cantilever-PLL feedback loop to confirm sufficient measurement bandwidth (in case of errors in setting the parameters). That transfer function relates the measured cantilever-PLL oscillation frequency to a cantilever's time-dependent intrinsic resonant frequency. Frequency data for each cantilever are collected at 500 Hz, and may be transmitted from the FPGA to a computer. The device may be placed on a copper heat sink/source connected to a heated water bath, maintained at 37 degrees C. The sample is loaded into the device from vials pressurized under air or air with 5% CO2 through 0.009 inch inner-diameter fluorinated ethylene propylene (FEP) tubing.

The pressurized vials may be seated in a temperature-controlled sample-holder throughout the measurement. FEP tubing allows the device to be flushed with piranha solution for cleaning, as piranha will damage most non-fluorinated plastics. To measure a sample of cells, the device may initially be flushed with filtered media, and then the sample may be flushed into one bypass channel. On large-channel devices, between one and two psi may be applied across the entire array, yielding flow rates on the order of 0.5 nL/s (the array's calculated fluidic resistance is approximately 3×10\hat{ }16 Pa/(m3/s). For small-channel devices, 4-5 psi may be applied across the array, yielding flow rates around 0.1 nL/s. Additionally, every several minutes new sample may be flushed into the input bypass channel to prevent particles and cells from settling in the tubing and device. Between experiments, devices may be cleaned with either filtered 10% bleach or piranha solution.

For the data analysis, the recorded frequency signals from each cantilever are rescaled by applying a rough correction for the different sensitivities of the cantilevers. Cantilevers differing in only their lengths should have mass sensitivities proportional to their resonant frequencies to the power three-halves. Therefore each frequency signal is divided by its carrier frequency to the power three-halves such that the signals are of similar magnitude. To detect peaks, the data are filtered with a low pass filter, followed by a nonlinear high pass filter (subtracting the results of a moving quantile filter from the data). Peak locations are found as local minima that occur below a user-defined threshold. After finding the peak locations, the peak heights may be estimated by fitting the surrounding baseline signal (to account for a possible slope in the baseline that was not rejected by the high pass filter), fitting the region surrounding the local minima with a fourth-order polynomial, and finding the maximum difference between the predicted baseline and the local minima polynomial fit. Identifying the peaks corresponding to calibration particles allows one to estimate the mass sensitivity for each cantilever, such that the modal mass for the particles is equal to the expected modal mass.

Peaks at different cantilevers that originate from the same cell are matched up to extract single-cell growth information. The serial SMR array and can measure live cells.

Certain embodiments include devices with piezo-resistors doped into the base of each cantilever, which are wired in parallel and their combined resistance measured via a Wheatstone bridge-based amplifier. The resulting deflection signal, which consists of the sum of k signals from the cantilever array, goes to an array of k phase-locked loops (PLLs) where each PLL locks to the unique resonant frequency of a single cantilever. Therefore there is a one to one pairing between cantilevers and PLLs. Each PLL determines its assigned cantilever's resonant frequency by demodulating its deflection signal and then generates a sinusoidal drive signal at that frequency. The drive signals from each PLL are then summed and used to drive a single piezo actuator positioned directly underneath the chip, completing the feedback loop. Each PLL is configured such that it will track its cantilever's resonant frequency with a bandwidth of 50 or 100 Hz. After acquiring the frequency signals for each cantilever, the signals are converted to mass units via each cantilever's sensitivity (Hz/pg), which is known precisely.

Various embodiments of SMR and sSMR instruments, as well as methods of use, include those instruments/devices manufactured by Innovative Micro Technology (Santa Barbara, Calif.) and described in U.S. Pat. Nos. 8,418,535 and 9,132,294, the contents of each of which are hereby incorporated by reference in their entirety.

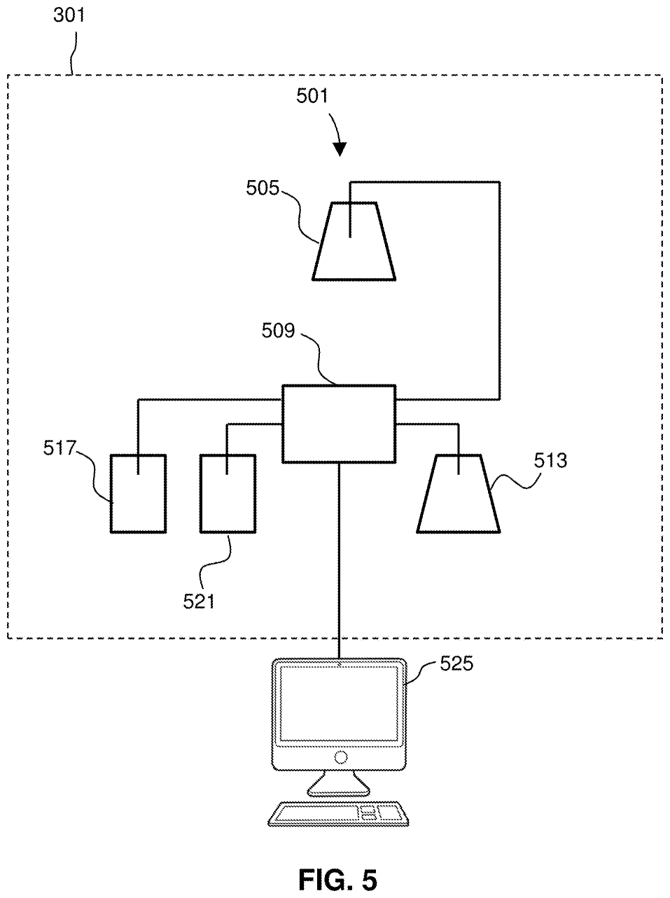

FIG. 5 shows a schematic diagram of an SMR detection system 501. As shown, a sample 505 (i.e., one or more live cells provided in a fluid medium) may be introduced to the SMR 509 of an instrument 301. As shown, the sample 505 and a buffer solution 513 may be provided to the SMR. The system 501 further includes an upper bypass channel collection outlet/reservoir 517 and lower bypass channel collection outlet/reservoir 521. The SMR 509 is configured to measure a functional biomarker of one or more live cells 505 flowing therethrough, such as density or mass of the sample, and transmit such measurements to a computer 525 that is communicatively coupled to the SMR 509, specifically communicatively coupled to the instrument 301. The computer 525 may be used for analysis and reporting of results. In some embodiments, a system for the functional biomarker measurement instrument may include additional analytical techniques, as will be described in greater detail herein. The computer 525 may further comprise a server and storage. Any of the elements in the SMR detection system 501 may interoperate via a network. The SMR 409 may include its own on-board computer. The computer 525 may include one or more processors and memory as well as an input/output mechanism.

Upon passing through the instrument 301, namely the exemplary flow path of a suspended microchannel or the flow path of the sSMR array 401, the cells remains viable and can be isolated downstream from the instrument 301 and are available to undergo the subsequent assays. The method further includes performing one or more additional assays on the live cells, either concurrently with the initial assay, or downstream from the first assay, to obtain further data associated with the live cells, such as additional functional data and/or genomic data.

It should be noted that methods of the disclosure include performing one or more additional assays on the live cells, either concurrently with the first assay, or downstream from the first assay, to obtain further functional or genetic data. In some embodiments, the second assay is performed on the live cells having undergone the first assay, which allows for data obtained from the first and second assays to be linked at a single-cell level, as opposed to a population level.

The one or more additional assays allow for single-cell analysis, including, for example, genome sequencing, single-cell transcriptomics, single-cell proteomics, and single-cell metabolomics.

Genome sequencing is generally the process of determining the order of nucleotides in DNA. It includes any method or technology that is used to determine the order of the four bases: adenine, guanine, cytosine, and thymine. Single cell DNA genome sequencing involves isolating a single cell, performing whole genome amplification (WGA), constructing sequencing libraries, and then sequencing the DNA using a next-generation sequencer (e.g., Illumina, Ion Torrent, etc.). Single cell genome sequencing is particularly of interest in the field of cancer study, as cancer cells are constantly mutating and it is of great interest to observer how cancers evolve at the genetic level. For example, single cell genome sequencing allowing for patterns of somatic mutations and copy number aberration to be observed.

Single-cell transcriptomics examines the gene expression level of individual cells in a given population by simultaneously measuring the messenger RNA (mRNA) concentration of hundreds to thousands of genes.

The purpose of single cell transcriptomics is to determine what genes are being expressed in each cell. The transcriptome is often used to quantify the gene expression instead of the proteome because of the difficulty currently associated with amplifying protein levels. Single-cell transcriptomics uses sequencing techniques similar to single cell genomics or direct detection using fluorescence in situ hybridization. The first step in quantifying the transcriptome is to convert RNA to cDNA using reverse transcriptase so that the contents of the cell can be sequenced using NGS methods, similar to what is done in single-cell genomics. Once converted, cDNA undergoes whole genome amplification (WGA), and then sequencing is performed. Alternatively, fluorescent compounds attached to RNA hybridization probes may be used to identify specific sequences and sequential application of different RNA probes will build up a comprehensive transcriptome.

Single cell transcriptomics can be used for various studies, such as, for example, gene dynamics, RNA splicing, and cell typing. Gene dynamics are usually studied to determine what changes in gene expression effect different cell characteristics. For example, this type of transcriptomic analysis has often been used to study embryonic development. RNA splicing studies are focused on understanding the regulation of different transcript isoforms. Single cell transcriptomics has also been used for cell typing, where the genes expressed in a cell are used to identify types of cells.

Single-cell proteomics is the study of proteomes (the entire complement of proteins that is or can be expressed by a cell, tissue, or organism) and their functions. The purpose of studying the proteome is to better understand the activity of cells at the single cells level. Since proteins are responsible for determining how the cell acts, understanding the proteome of single cell gives the best understanding of how a cell operates, and how gene expression changes in a cell due to different environmental stimuli. Although transcriptomics has the same purpose as proteomics it is not as accurate at determining gene expression in cells as it does not take into account post-transcriptional regulation.

There are three major approaches to single-cell proteomics: antibody based methods; fluorescent protein based methods; and mass-spectroscopy based methods. The antibody based methods use designed antibodies to bind to proteins of interest. These antibodies can be bound to fluorescent molecules such as quantum dots or isotopes that can be resolved by mass spectrometry. Since different colored quantum dots or different isotopes are attached to different antibodies it is possible to identify multiple different proteins in a single cell. Rare metal isotopes attached to antibodies, not normally found in cells or tissues, can be detected by mass spectrometry for simultaneous and sensitive identification of proteins. Another antibody based method converts protein levels to DNA levels. The conversion to DNA makes it possible to amplify protein levels and use NGS to quantify proteins. To do this, two antibodies are designed for each protein needed to be quantified. The two antibodies are then modified to have single stranded DNA connected to them that are complimentary. When the two antibodies bind to a protein the complimentary strands will anneal and produce a double stranded piece of DNA that can then be amplified using PCR. Each pair of antibodies designed for one protein is tagged with a different DNA sequence. The DNA amplified from PCR can then be sequenced, and the protein levels quantified.

In mass spectroscopy-based proteomics, there are three major steps needed for peptide identification: sample preparation; separation of peptides; and identification of peptides. Several groups have focused on oocytes or very early cleavage-stage cells since these cells are unusually large and provide enough material for analysis. Another approach, single cell proteomics by mass spectrometry (SCoPE-MS) has quantified thousands of proteins in mammalian cells with typical cell sizes (diameter of 10-15 μm) by combining carrier-cells and single-cell barcoding. Multiple methods exist to isolate the peptides for analysis. These include using filter aided sample preparation, the use of magnetic beads, or using a series of reagents and centrifuging steps. The separation of differently sized proteins can be accomplished by using capillary electrophoresis (CE) or liquid chromatograph (LC) (using liquid chromatography with mass spectroscopy is also known as LC-MS). This step gives order to the peptides before quantification using tandem mass-spectroscopy (MS/MS). The major difference between quantification methods is some use labels on the peptides such as tandem mass tags (TMT) or dimethyl labels which are used to identify which cell a certain protein came from (proteins coming from each cell have a different label) while others use not labels (quantify cells individually). The mass spectroscopy data is then analyzed by running data through databases that convert the information about peptides identified to quantification of protein levels. These methods are very similar to those used to quantify the proteome of bulk cells, with modifications to accommodate the very small sample volume. Improvements in sample preparation, mass-spec methods and data analysis can increase the sensitivity and throughput by orders of magnitude.

Single-cell metabolomics is study of chemical processes involving metabolites, the small molecule intermediates and products of metabolism, within cells. In particular, the purpose of single cell metabolomics is to gain a better understanding at the molecular level of major biological topics such as: cancer, stem cells, aging, as well as the development of drug resistance. In general the focus of metabolomics is mostly on understanding how cells deal with environmental stresses at the molecular level, and to give a more dynamic understanding of cellular functions. Accordingly, single cell metabolomics involves the study of a metabolome, which represents the complete set of metabolites in a biological cell, which are the end products of cellular processes. As generally understood, mRNA gene expression data and proteomic analyses reveal the set of gene products being produced in the cell, data that represents one aspect of cellular function. Conversely, metabolic profiling can give an instantaneous snapshot of the physiology of that cell, and thus, metabolomics provides a direct functional readout of the physiological state of an organism.

There are four major methods used to quantify the metabolome of single cells: fluorescence-based detection, fluorescence biosensors, FRET biosensors, and mass spectroscopy. The fluorescence-based detection, fluorescence biosensors, and FRET biosensors methods each use fluorescence microscopy to detect molecules in a cell. Such assays use small fluorescent tags attached to molecules of interest. However, it has been found that use of fluorescent tags may be too invasive for single cell metabolomics, and alters the activity of the metabolites. As such, the current solution to this problem is to use fluorescent proteins which will act as metabolite detectors, fluorescing whenever they bind to a metabolite of interest.

Mass spectroscopy is becoming the most frequently used method for single cell metabolomics, as there is no need to develop fluorescent proteins for all molecules of interest, and it is capable of detecting metabolites in the femtomole range. Similar to the methods discussed in proteomics, there has also been success in combining mass spectroscopy with separation techniques such as capillary electrophoresis to quantify metabolites. Another method utilizes capillary micro-sampling combined with mass spectrometry and ion mobility separation, which has been demonstrated to enhance the molecular coverage and ion separation for single cell metabolomics.

In other embodiments, the one or more additional assays may include flow cytometry to analyze physical and/or chemical characteristics of the one or more cells, including the detection of biomarkers. For example, a flow cytometer may be used to detect and measure chemical characteristics of cells by suspending the cells in a fluid, injecting the cells in the instrument, and flowing one cell at a time through a laser. The fluorescence can be measured to determine various properties of single particles, which are usually cells. Up to thousands of particles per second can be analyzed as they pass through the liquid stream. Examples of the properties measured include the particle's relative granularity, size and fluorescence intensity as well as its internal complexity. An optical-to-electronic coupling system is used to record the way in which the particle emits fluorescence and scatters incident light from the laser. Any suitable instrument may be used including for example one of the cell-sorting flow cytometry instruments sold under the trademarks FACSARIAIII by BD Biosciences, MOFLO XDP sold by Beckman Coulter, S3E sold by Bio-Rad, or VIVA G1 sold by Cytonome. For example, certain embodiments may use the cell sorting instrument sold under the trademark S3E cell sorter by Bio-Rad (Hercules, Calif.).

Accordingly, in one embodiment, performing 113 the second assay may include sequencing nucleic acid from the one or more live cells having undergone the first assay to produce sequence data. In order to perform nucleic acid sequencing, methods of the disclosure further include extracting nucleic acid from the one or more live cells having undergone the first analysis for a downstream sequencing step.

Isolation, extraction or derivation of genomic nucleic acids may be performed by methods known in the art. Isolating nucleic acid from a biological sample generally includes treating a biological sample in such a manner that genomic nucleic acids present in the sample are extracted and made available for analysis. Generally, nucleic acids are extracted using techniques such as those described in Green & Sambrook, 2012, Molecular Cloning: A Laboratory Manual 4 edition, Cold Spring Harbor Laboratory Press, Cold Spring Harbor, N.Y. (2028 pages), the contents of which are incorporated by reference herein. A kit may be used to extract DNA from tissues and bodily fluids and certain such kits are commercially available from, for example, BD Biosciences Clontech (Palo Alto, Calif.), Epicentre Technologies (Madison, Wis.), Gentra Systems, Inc. (Minneapolis, Minn.), and Qiagen Inc. (Valencia, Calif.). User guides that describe protocols are usually included in such kits.

It may be useful to lyse cells, isolate genomic nucleic acid, and optionally amplify nucleic acid. Amplification may be by polymerase chain reaction (PCR) as described in Dieffenbach, PCR Primer, a Laboratory Manual, 1995, Cold Spring Harbor Press, Plainview, N.Y., U.S. Pat. Nos. 4,683,195 and 4,683,202, all incorporated by reference. Nucleic acid may further be subject to analysis by sequencing.

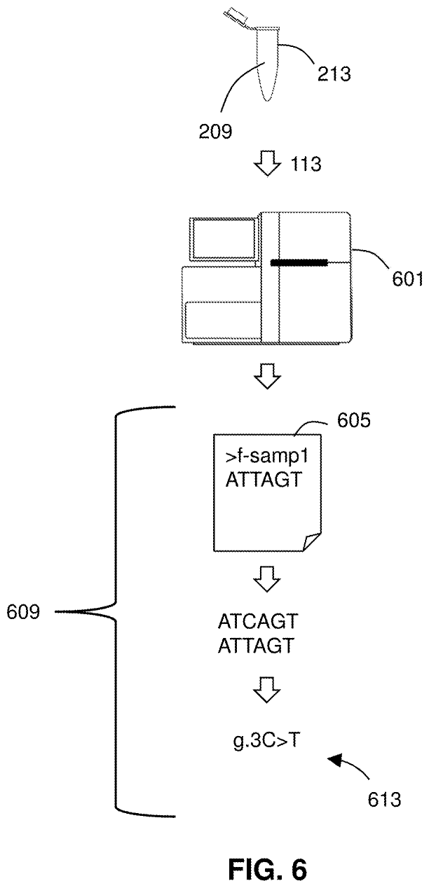

FIG. 6 diagrams a sequencing workflow according to certain embodiments. As shown, the method includes performing 113 a second assay on the one or more live cells having undergone the first assay (i.e., sample 209 of live cells collected from the device 301), wherein the second assay includes sequencing nucleic acid from the one or more live cells (from sample 209) using a sequencing instrument 601 to produce sequence reads 605. The method may further include analyzing 117 the sequence data (as well as the measured cancer biomarker from the first assay). For example, the analyzing 117 may include detecting one or more polymorphisms in the sequence data. Additionally, or alternatively, analyzing 117 may include mapping unique sequence reads to a reference to determine sub-chromosomal copy number variation or aneuploidy. Additionally, or alternatively, analyzing 117 may include determining expression levels in the one or more live cells. In some embodiments, analyzing 117 may further include determining tumor mutational burden (TMB). The TMB may be is determined by mapping sequence reads to a reference genome, identifying differences between the reads and the reference, and adding the identified difference to a mutation count. In some embodiments, the functional cancer biomarker measured in the first assay may include mass and/or mass change, wherein the functional cancer biomarker may be measured after administration of a checkpoint inhibitor. Yet still, in some embodiments, the functional cancer biomarker may include a mass or change in mass of a live cancer-related immune cell isolated from the same sample as the live cancer cell, such that the method may further include correlating the cancer-related immune cell biophysical data with the TMB data of the live cancer cell to generate composite biomarker indicating a stage or progression of the cancer.

In some embodiments, analyzing 117 includes analyzing sequence data from a plurality of different cells from a sample from the patient, assigning the cells to clonal groups based on the sequence data, and measuring the functional cancer biomarker for cells from specific clonal groups. In some embodiments, the functional cancer biomarker measured in the first assay may include a mass accumulation rate (MAR). As such, in some embodiments, analyzing 117 further includes identifying mutations exclusively present in clonal groups with the highest mass accumulation rate(s) as putative driver mutations. In some embodiments, analyzing 117 includes identifying mutations whose presence does not correlate with mass accumulation rate as passenger mutations.

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

A sequencing technique that can be used includes, for example, Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which is incorporated by reference in their entirety.

Sequencing produces a plurality of sequence reads 605. Sequence reads 605 generally include sequences of nucleotide data wherein read length may be associated with sequencing technology. Sequence reads 605 can be stored in any suitable file format including, for example, VCF files, FASTA files or FASTQ files, as are known to those of skill in the art. In some embodiments, PCR product is pooled and sequenced (e.g., on an Illumina HiSeq 2000). Raw .bcl files are converted to qseq files using bclConverter (Illumina). FASTQ files are generated by “de-barcoding” genomic reads using the associated barcode reads; reads for which barcodes yield no exact match to an expected barcode, or contain one or more low-quality base calls, may be discarded. Reads may be stored in any suitable format such as, for example, FASTA or FASTQ format.

The sequence reads may be analyzed to identify structural abnormalities, copy number variants, microdeletions, or duplications. In some embodiments, the sequence reads 605 are analyzed to identify sub chromosomal copy number alteration or an aneuploidy. The analysis may include variant calling 609, i.e., the analysis of sequence reads 605 to identify small mutations such as polymorphisms or small indels. To identify small mutations, reads may be mapped to a reference using assembly and alignment techniques known in the art or developed for use in the workflow. See U.S. Pat. Nos. 8,209,130; 8,165,821; 7,809,509; 6,223,128; U.S. Pub. 2011/0257889; and U.S. Pub. 2009/0318310, the contents of each of which are hereby incorporated by reference in their entirety. Sequence assembly or mapping may employ assembly steps, alignment steps, or both. Assembly can be implemented, for example, by the program ‘The Short Sequence Assembly by k-mer search and 3′ read Extension’ (SSAKE), from Canada's Michael Smith Genome Sciences Centre (Vancouver, B.C., CA) (see, e.g., Warren et al., 2007, Assembling millions of short DNA sequences using SSAKE, Bioinformatics, 23:500-501). SSAKE cycles through a table of reads and searches a prefix tree for the longest possible overlap between any two sequences. SSAKE clusters reads into contigs.

Aligned or assembled sequence reads may be analyzed for the presence of variants 613, e.g., mutations described, or “called” as variants of a given reference. Mutation calling is described in U.S. Pub. 2013/0268474. In certain embodiments, analyzing the reads includes assembling the sequence reads and then genotyping the assembled reads. In certain embodiments, reads are aligned to hg18 on a per-sample basis using Burrows-Wheeler Aligner version 0.5.7 for short alignments, and genotype calls are made using Genome Analysis Toolkit. See McKenna et al., 2010, The Genome Analysis Toolkit: a MapReduce framework for analyzing next-generation DNA sequencing data, Genome Res 20(9):1297-1303 (aka the GATK program).

Mapping sequence reads to a reference, by whatever strategy, may produce output such as a text file or an XML file containing sequence data such as a sequence of the nucleic acid aligned to a sequence of the reference genome. In certain embodiments mapping reads to a reference produces results stored in SAM or BAM file and such results may contain coordinates or a string describing one or more mutations in the subject nucleic acid relative to the reference genome. Alignment strings known in the art include Simple UnGapped Alignment Report (SUGAR), Verbose Useful Labeled Gapped Alignment Report (VULGAR), and Compact Idiosyncratic Gapped Alignment Report (CIGAR). See Ning et al., 2001, SSAHA: A fast search method for large DNA database, Genome Research 11(10):1725-9. These strings are implemented, for example, in the Exonerate sequence alignment software from the European Bioinformatics Institute (Hinxton, UK).

In some embodiments, a sequence alignment is produced—such as, for example, a sequence alignment map (SAM) or binary alignment map (BAM) file—comprising a CIGAR string (the SAM format is described, e.g., in Li, et al., The Sequence Alignment/Map format and SAMtools, Bioinformatics, 2009, 25(16):2078-9). In some embodiments, CIGAR displays or includes gapped alignments one-per-line. CIGAR is a compressed pairwise alignment format reported as a CIGAR string. A CIGAR string is useful for representing long (e.g. genomic) pairwise alignments. A CIGAR string is used in SAM format to represent alignments of reads to a reference genome sequence.

Output from mapping may be stored in a SAM or BAM file, in a variant call format (VCF) file, or other format. In an illustrative embodiment, output is stored in a VCF file. A typical VCF file will include a header section and a data section. The header contains an arbitrary number of meta-information lines, each starting with characters ‘##’, and a TAB delimited field definition line starting with a single ‘#’ character. The field definition line names eight mandatory columns and the body section contains lines of data populating the columns defined by the field definition line. The VCF format is described in Danecek et al., 2011, The variant call format and VCFtools, Bioinformatics 27(15):2156-2158.

The data contained in a VCF file represents the variants 613, or mutations, that are found in the nucleic acid that was obtained from the sample from the patient and sequenced. In its original sense, mutation refers to a change in genetic information and has come to refer to the present genotype that results from a mutation. As is known in the art, mutations include different types of mutations such as substitutions, insertions or deletions (INDELs), translocations, inversions, chromosomal abnormalities, and others. Variant can be taken to be roughly synonymous to mutation but referring to a genotype being described in comparison or with reference to a reference genotype or genome. For example as used in bioinformatics variant describes a genotype feature in comparison to a reference such as the human genome (e.g., hg18 or hg19 which may be taken as a wild type). Methods described herein may generate data representing one or more mutations, or variant calls 613.

A description of a mutation may be provided according to a systematic nomenclature, e.g., a substitution name starts with a number followed by a “from to” markup 413 (199A>G shows that at position 199 of the reference sequence, A is replaced by a G). See den Dunnen & Antonarakis, 2003, Mutation Nomenclature, Curr Prot Hum Genet 7.13.1-7.13.8, incorporated by reference.

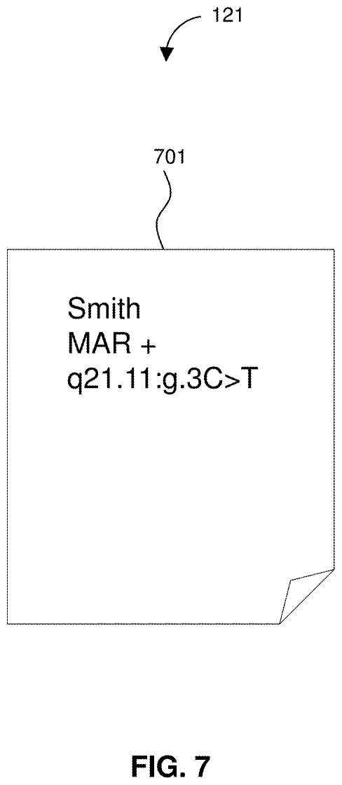

FIG. 7 shows a report 701 as may be provided. The report 701 may include any suitable patient information including identity along with information related to the cancer evaluation, including, but not limited to, whether the sample tested positive for cancer, a determination of a stage or progression of cancer, and a customized treatment plan tailored to an individual patient's cancer diagnosis. In some embodiments, the report 701 describes one or more genetic sequence alterations and the measured functional biomarker (i.e., mass, change in mass, mass accumulation rate, etc.) in the live cells from the patient. The report 701 may be anonymized (e.g., according to an encoded patient ID). The report 701 may be provided on paper or may be stored or transmitted electronically, e.g., as a PDF or XML document. The report may include any clinically-significant biophysical properties (e.g., mass, change in mass, mass accumulation rate (MAR)) and/or genetic information determined from the isolated cells. For patient reporting or notification, systems and methods of the invention may be used to retrieve medical/clinical information from an outside database. The outside database may be a clinical decision support system such as UP2DATE by Wolters-Kluwer. Any suitable clinical decision support resources may be included in the outside database that is queried by the system. Other suitable resources include the medical reference resource sold under the name EPOCRATES by Athena Health (Watertown, Mass.). Other clinical decision support (CDS) resources that may be accessed may include the PREDICT (Pharmacogenomic Resource for Enhanced Decisions in Care and Treatment) project, the CLIPMERGE (Clinical Implementation of Personalized Medicine through Electronic Health Records and Genomics) program, and the SMART (Substitutable Medical Apps Reusable Technologies) Genomics Adviser. Thus the method 101 may include analyzing 117 the sequence data and the measured functional biomarker to determine a stage or progression of the cancer.

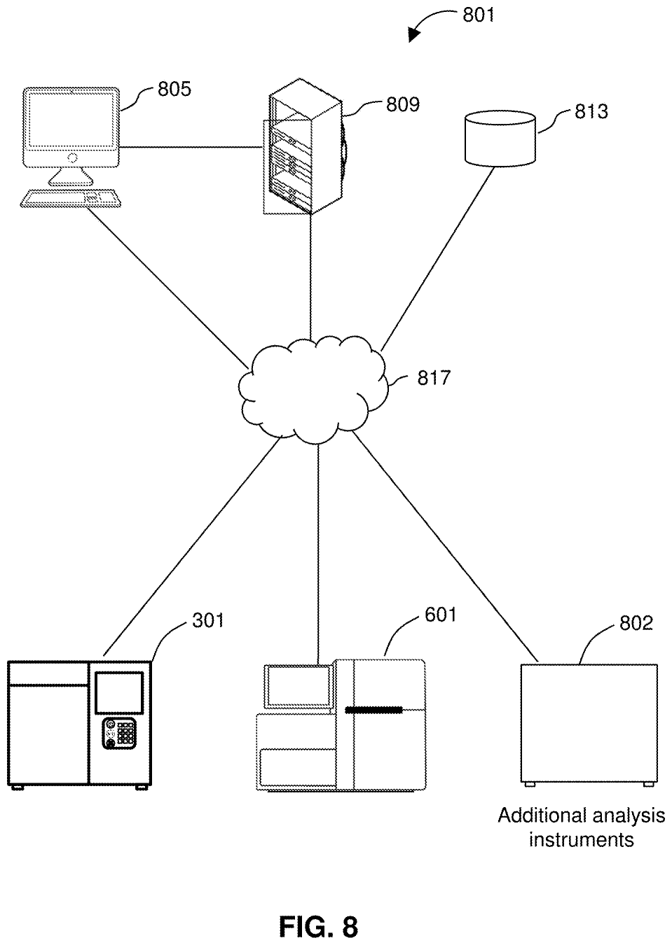

FIG. 8 is a block diagram of a system 801 according to embodiments of the invention. The system 801 may include one or more of an instrument 301 comprising a suspended microchannel resonator (SMR), a sequencing instrument 601, and any additional analysis instruments 801 for performing additional assays on the one or more cells downstream of the initial assay performed by instrument 301, a computer 805, a server 809, and storage 813. Any of those elements may interoperate via a network 817. Any one of the instruments 301, 601, and 802 may include its own on-board computer. The computer 805 may include one or more processors and memory as well as an input/output mechanism. Where methods of the invention employ a client/server architecture, steps of methods of the invention may be performed using the server 809, which includes one or more of processors and memory, capable of obtaining data, instructions, etc., or providing results via an interface module or providing results as a file. The server 809 may be provided by a single or multiple computer devices, such as the rack-mounted computers sold under the trademark BLADE by Hitachi. The server 809 may be provided as a set of servers located on or off-site or both. The server 809 may be owned or provided as a service. The server 809 or the storage 813 may be provided wholly or in-part as a cloud-based resources such as Amazon Web Services or Google. The inclusion of cloud resources may be beneficial as the available hardware scales up and down immediately with demand. The actual processors—the specific silicon chips—performing a computation task can change arbitrarily as information processing scales up or down. In an embodiment, the server 809 includes one or a plurality of local units working in conjunction with a cloud resource (where local means not-cloud and includes or off-site). The server 809 may be engaged over the network 817 by the computer 805 and either or both may engage the outside database (not shown).

In system 801, each computer preferably includes at least one processor coupled to a memory and at least one input/output (I/O) mechanism. A processor will generally include a chip, such as a single core or multi-core chip, to provide a central processing unit (CPU). A process may be provided by a chip from Intel or AMD.

Memory can include one or more machine-readable devices on which is stored data or instructions (e.g., software) which, when executed by the processor(s), cause a system to perform methods of the invention. Memory preferably includes any combination of RAM, hard drives, solid-state memories (e.g., subscriber identity module (SIM) card, secure digital card (SD card), micro SD card, or solid-state drive (SSD)), optical and magnetic media, and/or any other tangible storage medium or media. A computer of the invention will generally include one or more I/O device such as, for example, one or more of a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Any of the software can be physically located at various positions, including being distributed such that portions of the functions are implemented at different physical locations.

The system 701 or components of system 701 may be used to perform methods described herein. Instructions for any method step may be stored in memory and a processor may execute those instructions.

The system 801 thus includes at least one computer (and optionally one or more instruments) operable to obtain one or more live cells isolated from a sample of a patient, wherein the one or more live cells comprise at least one of a cancer cell and a cancer-related immune cell. The system 801 is further operable to perform a first assay on the one or more live cells, wherein the first assay comprises measuring a functional cancer biomarker in the one or more live cells. The system 801 is further operable to perform a second assay on the one or more live cells having undergone the first assay. The system 801 is further operable to analyze data from the second assay and the measured cancer biomarker to determine at least a stage or progression of the cancer. Using the computer 801, the system is operable to provide a report comprising any suitable patient information including identity along with information related to the cancer evaluation, including, but not limited to, specific data associated with the first and second assays, a determination of a stage or progression of cancer, and personalized treatment tailored to an individual patient's cancer.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for evaluating cancer, the method comprising:
obtaining one or more live cells isolated from a sample of a patient;
performing a first assay to measure a functional biomarker in the one or more live cells;
performing a second assay on the one or more live cells having undergone the first assay; and
analyzing data from the second assay and the measured biomarker to determine a stage or progression of the cancer.

2. The method of claim 1, wherein the one or more live cells include cancer cells or immune cells.

3. The method of claim 1, wherein the second assay is selected from the group consisting of genome sequencing, single cell transcriptomics, single cell proteomics, and single cell metabolomics.

4. The method of claim 1, wherein:
the performing the second assay step comprises sequencing nucleic acid from the one or more live cells having undergone the first assay to produce sequence data; and
the analyzing step comprises analyzing the sequence data.

5. The method of claim 4, wherein the analyzing step comprises detecting one or more polymorphisms in the sequence data.

6. The method of claim 4, wherein the analyzing step comprises mapping unique sequence reads to a reference to determine sub-chromosomal copy number variation or aneuploidy.

7. The method of claim 4, wherein the analyzing step comprises determining tumor mutational burden (TMB).

8. The method of claim 7, wherein TMB is determined by mapping sequence reads to a reference genome, identifying differences between the reads and the reference, and adding the identified difference to a mutation count.

9. The method of claim 1, wherein the functional biomarker includes mass or mass change.

10. The method of claim 1, wherein the functional biomarker is measured after administration of a checkpoint inhibitor.

11. The method of claim 4, further comprising analyzing sequence data from a plurality of different cells from a sample from the patient, assigning the cells to clonal groups based on the sequence data, and measuring the functional cancer biomarker for cells from specific clonal groups.

12. The method of claim 11, wherein the functional cancer biomarker comprises a mass accumulation rate.

13. The method of claim 11, further comprising identifying mutations exclusively present in clonal groups with the highest mass accumulation rate(s) as putative driver mutations.

14. The method of claim 11, further comprising identifying mutations whose presence does not correlate with mass accumulation rate as passenger mutations.

15. The method of claim 3, further comprising providing a report that describes one or more genetic sequence alterations and the measured cancer biomarker in the live cells from the patient.

16. The method of claim 1, wherein the functional biomarker comprises a mass or change in mass of a cell.

17. The method of claim 16, wherein the measuring step is performed using a suspended microchannel resonator.

18. The method of claim 1, wherein the analyzing step comprises determining tumor mutational burden (TMB) of a live cancer cell isolated from the sample.

19. The method of claim 18, wherein the functional biomarker comprises a mass or change in mass of the live cancer cell.

20. The method of claim 19, further comprising correlating the mass change with the TMB to generate composite biomarker indicating a stage or progression of the cancer.
